(19) United States
(12) Patent Application Publication
(10) Pub. No.: US 2009/0163451 A1
(43) Pub. Date: Jun. 25, 2009

(54) METHODS FOR TREATING VISCERAL PAIN

(76) Inventors: Frank Porreca, Tucson, AR (US); Louis P. Vera-Portocarrero, Tucson, AR (US)

Correspondence Address: CLARK & ELBING LLP, 101 FEDERAL STREET, BOSTON, MA, 02110, US

(21) Appl. No.: 12/272,734

(22) Filed: Nov. 17, 2008

Related U.S. Application Data
(60) Provisional application No. 60/988,729, filed on Nov. 16, 2007.

(51) Int. Cl. A61K 31/60; A61K 31/404; A61K 31/4196; A61K 31/454; A61K 31/422; A61K 31/403; A61K 31/415; A61K 31/444; A61K 31/42; A61K 31/4525; A61K 31/4535; A61K 31/55; A61K 31/5513; A61K 31/439; A61K 31/4178; A61K 31/46; A61K 31/473; A61P 29/00; A61K 31/445

(52) U.S. Cl. 514/165; 514/415; 514/383; 514/323; 514/376; 514/414; 514/411; 514/406; 514/334; 514/378; 514/321; 514/326; 514/217; 514/221; 514/220; 514/294; 514/304; 514/397; 514/296; 514/317

(57) ABSTRACT

The invention features methods of treating visceral pain in humans by administering an effective amount of a 5HT_{1B} or 5HT_{1D} receptor agonist, (e.g., a triptan). These methods can be used, for example, to treat a human suffering from visceral pain secondary to an underlying disease of a visceral organ, such as pancreatitis. Visceral pain treatable by the methods of the invention may also be secondary to a disease of the liver, kidney, ovary, uterus, bladder, bowel, stomach, esophagus, duodenum, intestine, colon, spleen, pancreas, appendix, heart, or peritoneum.

### CROSS-REFERENCE TO RELATED APPLICATIONS

This application claims benefit of U.S. Provisional Application No. 60/988,729, filed on Nov. 16, 2007, which is hereby incorporated by reference.

### STATEMENT AS TO FEDERALLY SPONSORED RESEARCH

This research has been sponsored in part by NIH grant number PO1 DA 06284-01. The government has certain rights to the invention.

### BACKGROUND OF THE INVENTION

In general, the invention relates to the treatment of visceral pain. Visceral pain is of great concern to the medical community because the onset of visceral pain is a leading cause of patient visits to the clinic and because effective treatments for visceral pain are limited. Visceral pain is distinct from somatic pain and is generally described as pain that originates from the body's internal cavities or organs. Visceral pain has five important clinical and sensory characteristics: (1) it is not evoked from all visceral organs (e.g., liver or lung); (2) it is not always elicited by visceral injury (e.g., cutting an intestine does not evoke pain); (3) it is diffuse; (4) it may be referred to other locations; and (5) it may be associated with other autonomic and motor reflexes (e.g., nausea, lower-back muscle tension from renal colic) (Lancet 1999, 353, 2145-48).

Several theories have been proposed to explain the mechanisms of visceral pain. In the first theory, the viscera are innervated by separate classes of neurons, one concerned with autonomic regulation and the other with sensory phenomena such as pain. The second theory suggests a single homogeneous class of sensory receptors that are active at low frequencies (normal regulatory signals) or at high frequencies of activation (induced by intense pain signals). However, some studies indicate that the viscera is innervated by two classes of nociceptive sensory receptors: high threshold (mostly mechanical receptors found in heart, vein, lung, airways, esophagus, biliary system, small intestine, colon, ureter, airways, urinary bladder and uterus; activated by noxious stimuli) and low threshold intensity coding receptors that respond to innocuous and nocuous stimuli (heart, oesophagus, colon, urinary bladder and testes). Yet another theory suggests a component of afferent fibres that are normally unresponsive to stimuli (silent nociceptors) which can become activated or sensitized during inflammation. Once sensitized, these nociceptors respond to innocuous stimuli that normally occur in the internal organs, resulting in convergent inputs to the spinal cord and subsequent pain amplification by central mechanisms.

Previous studies have implicated the RVM (rostral ventral medulla) in descending modulation of visceral pain. Electrical stimulation of the RVM produces biphasic modulation of spinal cord responses to colorectal distention (CRD) and of CRD-induced nociceptive reflexes. Microinjection of lidocaine into the RVM reduced spontaneous activity and responses of spinal neurons to CRD. These studies were done on reflexes induced by acute visceral pain. The RVM also has a facilitatory role on persistent visceral pain. Microinjection of lidocaine into the RVM attenuated referred visceral hypersensitivity induced by pancreatic inflammation.

Two useful models for the study of visceral pain are pancreatitis and colonic hypersensitivity. Pain from pancreatitis can be referred to somatic structures in humans and in animal models. Thus, measuring the degree of referred somatic hypersensitivity has become a useful tool to investigate visceral hypersensitivity. Colonic hypersensitivity is a more recent model of visceral pain. This model mimics aspects of irritable bowel syndrome (IBS) as there is presence of visceral hypersensitivity without apparent injury as observed in IBS patients. In this model, measuring referred lumbar hypersensitivity is also a reliable measurement of visceral hypersensitivity. In IBS patients, the predominant complaint is pain, which can be referred to lumbar dermatomes.

Visceral pain is difficult to manage clinically and often requires the use of opiates. Although widely used, the severe dose-limiting adverse effects of opiates often result in diminished efficacy. Additionally, opiates carry the risk of abuse and physical dependence and induce constipation and other unwanted adverse effects, which diminish quality of life. For this reason, improved treatments for visceral pain are highly desirable.

### SUMMARY OF THE INVENTION

The invention features methods of treating visceral pain in humans by administering an effective amount of a 5HT_{1B} or 5HT_{1D} (i.e., serotonin receptor) receptor agonist. These methods can be used, for example, to treat a human suffering from visceral pain secondary to an underlying disease of a visceral organ, such as pancreatitis. Visceral pain treatable by the methods of the invention may also be secondary to a disease of the liver, kidney, ovary, uterus, bladder, bowel, stomach, esophagus, duodenum, intestine, colon, spleen, pancreas, appendix, heart, or peritoneum. Alternatively, the visceral pain may result from irritable bowel syndrome, inflammatory bowel syndrome, pancreatitis, diverticulitis, Crohn's disease, peritonitis, pericarditis, hepatitis, appendicitis, colitis, cholecystitis, gastroenteritis, endometriosis, dysmenorrhea, interstitial cystitis, upper gastrointestinal dyspepsia, renal colic, biliary colic, or infection of a visceral organ. Also included in the invention is the administration of an effective amount of a 5HT_{1B} or 5HT_{1D} receptor agonist to treat visceral pain resulting from a neoplasm, from injury, or from inflammatory or non-inflammatory diseases. In any of the above methods, 5HT_{1B} and 5HT_{1D} receptor agonists may be co-administered.

In certain embodiments, visceral pain is treated with a triptan. Particular embodiments of the invention include the use of sumatriptan, rizatriptan, naratriptan, zolmitriptan, eletriptan, almotriptan, or frovatriptan for the treatment of visceral pain.

In certain embodiments, the human has been diagnosed with visceral pain prior to administration of the 5HT_{1B} or 5HT_{1D} receptor agonist. In other embodiments, the human is not suffering from a migraine or a cluster headache.

The invention further features a method of treating visceral pain by the co-administration of a 5HT_{1B} or 5HT_{1D} receptor agonist with an analgesic. Exemplary analgesics include, without limitation, neurokinin antagonists, cholecystokinin (CCK) antagonists, opiates, paracetamol, or nonsteroidal anti-inflammatory drugs (NSAIDs). The NSAID may be, for example, aspirin, ibuprofen, naproxen, or a selective cyclooxygenase 2 (COX-2) inhibitor, such as celecoxib, etoricoxib, lumiracoxib, parecoxib, rofecoxib, or valdecoxib.

The invention also features the co-administration of a 5HT_{1B} or 5HT_{1D} receptor agonist with one or more additional agents selected from antidepressants, anxiolytics, antiemetics, amphetamines, NOS inhibitors, and anticonvulsants for the treatment of visceral pain. The antidepressant is, e.g., amitriptyline, desipramine, fluoxetine, paroxetine, venlafaxine, sertraline, escitalopram, citalopram, fluvoxamine, milnacipran, or duloxetine. The anxiolytic is, e.g., lorazepam, clonazepam, alprazolam and diazepam. The antiemetic is, e.g., dolasetron, granisetron, odansetron, tropisetron, or palonosetron. The amphetamine is, e.g., methylphenidate. The anticonvulsant is, e.g., gabapentin, valproate, or carbamazapine, for the treatment of visceral pain.

In certain embodiments, a 5HT_{1B} or 5HT_{1D} receptor agonist is co-administered with an agent selected from the agents of Table 1.

TABLE 1
Therapeutic agents useful in combination with compounds of the invention

| Class | Examples |
|---|---|
| Opiate | alfentanil, butorphanol, buprenorphine, codeine, dextromoramide, |
| | dextropropoxyphene, dezocine, dihydrocodeine, diphenoxylate, etorphine, fentanyl, |
| | hydrocodone, hydromorphone, ketobemidone, levorphanol, levomethadone, |
| | methadone, meptazinol, morphine, morphine-6-glucuronide, nalbuphine, naloxone, |
| | oxycodone, oxymorphone, pentazocine, pethidine, piritramide, remifentanil, |
| | sulfentanyl, tilidine, or tramadol |
| Antidepressant | citalopram, escitalopram, fluoxetine, fluvoxamine, paroxetine, or sertraline |
| (selective serotonin | |
| re-uptake inhibitor) | |
| Antidepressant | clomipramine, doxepin, imipramine, imipramine oxide, trimipramine, adinazolam, |
| (norepinephrine- | amiltriptylinoxide, amoxapine, desipramine, maprotiline, nortriptyline, |
| reuptake inhibitor) | protriptyline, amineptine, butriptyline, demexiptiline, dibenzepin, dimetacrine, |
| | dothiepin, fluacizine, iprindole, lofepramine, melitracen, metapramine, |
| | norclolipramine, noxiptilin, opipramol, perlapine, pizotyline, propizepine, |
| | quinupramine, reboxetine, atomoxetine, bupropion, reboxetine, or tianeptine |
| Antidepressant | duloxetine, milnacipran, mirtazapine, nefazodone, or venlafaxine |
| (dual serotonin/ | |
| norepinephrine | |
| reuptake inhibitor) | |
| Antidepressant | amiflamine, iproniazid, isocarboxazid, M-3-PPC (Draxis), moclobemide, pargyline, |
| (monoamine oxidase | phenelzine, tranylcypromine, or vanoxerine |
| inhibitor) | |
| Antidepressant | bazinaprine, befloxatone, brofaromine, cimoxatone, or clorgyline |
| (reversible | |
| monoamine oxidase | |
| type A inhibitor) | |
| Antidepressant | amitriptyline, clomipramine, desipramine, doxepin, imipramine, maprotiline, |
| (tricyclic) | nortryptyline, protriptyline, or trimipramine |
| Antidepressant | adinazolam, alaproclate, amineptine, amitriptyline/chlordiazepoxide combination, |
| (other) | atipamezole, azamianserin, bazinaprine, befuraline, bifemelane, binodaline, |
| | bipenamol, brofaromine, caroxazone, cericlamine, cianopramine, cimoxatone, |
| | citalopram, clemeprol, clovoxamine, dazepinil, deanol, demexiptiline, dibenzepin, |
| | dothiepin, droxidopa, enefexine, estazolam, etoperidone, femoxetine, fengabine, |
| | fezolamine, fluotracen, idazoxan, indalpine, indeloxazine, iprindole, levoprotiline, |
| | lithium, litoxetine; lofepramine, medifoxamine, metapramine, metralindole, |
| | mianserin, milnacipran, minaprine, mirtazapine, montirelin, nebracetam, nefopam, |
| | nialamide, nomifensine, norfluoxetine, orotirelin, oxaflozane, pinazepam, |
| | pirlindone, pizotyline, ritanserin, rolipram, sercloremine, setiptiline, sibutramine, |
| | sulbutiamine, sulpiride, teniloxazine, thozalinone, thymoliberin, tianeptine, |
| | tiflucarbine, trazodone, tofenacin, tofisopam, toloxatone, tomoxetine, veralipride, |
| | viloxazine, viqualine, zimelidine, or zometapine |
| Antiepileptic | carbamazepine, flupirtine, gabapentin, lamotrigine, oxcarbazepine, phenytoin, |
| | retigabine, topiramate, or valproate |
| Non-steroidal anti- | acemetacin, aspirin, celecoxib, deracoxib, diclofenac, diflunisal, ethenzamide, |
| inflammatory drug | etofenamate, etoricoxib, fenoprofen, flufenamic acid, flurbiprofen, lonazolac, |
| (NSAID) | lornoxicam, ibuprofen, indomethacin, isoxicam, kebuzone, ketoprofen, ketorolac, |
| | naproxen, nabumetone, niflumic acid, piroxicam, meclofenamic acid, mefenamic |
| | acid, meloxicam, metamizol, mofebutazone, oxyphenbutazone, parecoxib, |
| | phenidine, phenylbutazone, piroxicam, propacetamol, propyphenazone, rofecoxib, |
| | salicylamide, suprofen, sulindac, tiaprofenic acid, tolmetin, tenoxicam, valdecoxib, |
| | 4-(4-cyclohexyl-2-methyloxazol-5-yl)-2-fluorobenzenesulfonamide, N-[2- |
| | (cyclohexyloxy)-4-nitrophenyl]methanesulfonamide, 2-(3,4-difluorophenyl)-4-(3- |
| | hydroxy-3-methylbutoxy)-5-[4-(methylsulfonyl)phenyl]-3(2H)-pyridazinone, or 2- |
| | (3,5-difluorophenyl)-3-[4-(methylsulfonyl)phenyl]-2-cyclopenten-1-one). |
| Anti-inflammatory | aspirin, celecoxib, cortisone, deracoxib, diflunisal, etoricoxib, fenoprofen, |
| compounds | ibuprofen, ketoprofen, naproxen, prednisolone, sulindac, tolmetin, piroxicam, |
| | mefenamic acid, meloxicam, phenylbutazone, rofecoxib, suprofen, valdecoxib, 4- |
| | (4-cyclohexyl-2-methyloxazol-5-yl)-2-fluorobenzenesulfonamide, N-[2- |
| | (cyclohexyloxy)-4-nitrophenyl]methanesulfonamide, 2-(3,4-difluorophenyl)-4-(3- |
| | hydroxy-3-methylbutoxy)-5-[4-(methylsulfonyl)phenyl]-3(2H)-pyridazinone, or 2- |
| | (3,5-difluorophenyl)-3-[4-(methylsulfonyl)phenyl]-2-cyclopenten-1-one |
| N-methyl-D- | amantadine; aptiganel; besonprodil; budipine; conantokin G; delucemine; |
| aspartate antagonist | dexanabinol; dextromethorphan; dextropropoxyphen; felbamate; fluorofelbamate; |
| | gacyclidine; glycine; ipenoxazone; kaitocephalin; ketamine; ketobemidone; |
| | lanicemine; licostinel; midafotel; memantine; D-methadone; D-morphine; |
| | milnacipran; neramexane; orphenadrine; remacemide; sulfazocine; FPL-12,495 |
| | (racemide metabolite); topiramate; (αR)-α-amino-5-chloro-1-(phosphonomethyl)- |
| | 1H-benzimidazole-2-propanoic acid; 1-aminocyclopentane-carboxylic acid; [5- |
| | (aminomethyl)-2-[[[(5S)-9-chloro-2,3,6,7-tetrahydro-2,3-dioxo-1H-,5H- |
| | pyrido[1,2,3-de]quinoxalin-5-yl]acetyl]amino]phenoxy]-acetic acid; α-amino-2-(2- |
| | phosphonoethyl)-cyclohexanepropanoic acid; α-amino-4-(phosphonomethyl)- |
| | benzeneacetic acid; (3E)-2-amino-4-(phosphonomethyl)-3-heptenoic acid; 3-[(1E)- |
| | 2-carboxy-2-phenylethenyl]-4,6-dichloro-1H-indole-2-carboxylic acid; 8-chloro- |
| | 2,3-dihydropyridazino[4,5-b]quinoline-1,4-dione 5-oxide salt with 2-hydroxy- |
| | N,N,N-trimethyl-ethanaminium; N′-[2-chloro-5-(methylthio)phenyl]-N-methyl-N- |
| | [3-(methylthio)phenyl]-guanidine; N′-[2-chloro-5-(methylthio)phenyl]-N-methyl- |
| | N-[3-[(R)-methylsulfinyl]phenyl]-guanidine; 6-chloro-2,3,4,9-tetrahydro-9-methyl- |
| | 2,3-dioxo-1H-indeno[1,2-b]pyrazine-9-acetic acid; 7-chlorothiokynurenic acid; |
| | (3S,4aR,6S,8aR)-decahydro-6-(phosphonomethyl)-3-isoquinolinecarboxylic acid; (−)- |
| | 6,7-dichloro-1,4-dihydro-5-[3-(methoxymethyl)-5-(3-pyridinyl)-4-H-1,2,4-triazol- |
| | 4-yl]-2,3-quinoxalinedione; 4,6-dichloro-3-[(E)-(2-oxo-1-phenyl-3- |
| | pyrrolidinylidene)methyl]-1H-indole-2-carboxylic acid; (2R,4S)-rel-5,7-dichloro- |
| | 1,2,3,4-tetrahydro-4-[[(phenylamino)carbonyl]amino]-2-quinolinecarboxylic acid; |
| | (3R,4S)-rel-3,4-dihydro-3-[4-hydroxy-4-(phenylmethyl)-1-piperidinyl-]-2H-1- |
| | benzopyran-4,7-diol; 2-[(2,3-dihydro-1H-inden-2-yl)amino]-acetamide; 1,4- |
| | dihydro-6-methyl-5-[(methylamino)methyl]-7-nitro-2,3-quinoxalinedione; [2-(8,9- |
| | dioxo-2,6-diazabicyclo[5.2.0]non-1(7)-en-2-yl)ethyl]-phosphonicacid; (2R,6S)- |
| | 1,2,3,4,5,6-hexahydro-3-[(2S)-2-methoxypropyl]-6,11,11-trimethyl-2,6-methano-3- |
| | benzazocin-9-ol; 2-hydroxy-5-[[(pentafluorophenyl)methyl]amino]-benzoic acid; 1- |
| | [2-(4-hydroxyphenoxy)ethyl]-4-[(4-methylphenyl)methyl]-4-piperidinol; 1-[4-(1H- |
| | imidazol-4-yl)-3-butynyl]-4-(phenylmethyl)-piperidine; 2-methyl-6- |
| | (phenylethynyl)-pyridine; 3-(phosphonomethyl)-L-phenylalanine; or 3,6,7- |
| | tetrahydro-2,3-dioxo-N-phenyl-1H,5H-pyrido[1,2,3-de]quinoxaline-5-acetamide |

In any of the embodiments of the invention, the 5HT_{1B} or 5HT_{1D} receptor agonist may be admixed or formulated with a pharmaceutically acceptable carrier. The 5HT_{1B} or 5HT_{1D} receptor agonist may be administered by any suitable route, e.g., by intracolonic instillation.

In certain embodiments, the 5HT_{1B} or 5HT_{1D} receptor agonist directly binds 5HT_{1B} or 5HT_{1D} receptors.

### DEFINITIONS

As used herein, by a “5HT_{1B} agonist” and “5HT_{1D} agonist” are meant, respectively, an agent that enhances the activity of 5-hydroxytryptamine/serotonin receptors 1B and/or 1D, e.g., by directly binding and activating 5HT_{1B} or 5HT_{1D} receptors (e.g., as with a triptan) or by inhibiting reuptake of serotonin (e.g., as with an SSRI). Agonists of 5HT_{1B/1D} receptors include, but are not limited to, antidepressants or anxiolytics (e.g., citalopram), amphetamines (e.g., dextroamphetamine and levoamphetamine), antiemetics or anxiolytics (e.g., benzodiazepines), anticonvulsants (e.g., sodium valproate), and triptans (e.g., sumatriptan). An agonist of 5HT_{1B} receptors may also agonize 5HT_{1D} receptors; conversely, an agonist of 5HT_{1D} receptors may also agonize 5HT_{1B} receptors.

A “direct agonist” is a compound that directly binds to a receptor resulting in agonist activity.

By “analgesic” is meant any member of the diverse group of drugs used to relieve pain. Analgesic drugs act in various ways on the peripheral and central nervous systems. They include, but are not limited to, paracetamol (acetaminophen), the nonsteroidal anti-inflammatory drugs (NSAIDs), and opiate drugs such as morphine.

By “antidepressant” is meant any member of the diverse group of drugs used to relieve depression or dysthymia. Classes of antidepressants include selective serotonin reuptake inhibitors (SSRIs), serotonin-norepinephrine reuptake inhibitors (SNRIs), noradregnergic and specific serotonergic antidepressants (NASSAs), norepinephrine (noradrenaline) reuptake inhibitors (NRIs), norepinephrine-dopamine reuptake inhibitors, tricyclic antidepressants (TCAs), and monoamine oxidase inhibitors (MAOs). Examples of antidepressant agents include, but are not limited to, amitriptyline, citalopram, desipramine, duloxetine, escitalopram, fluoxetine, fluvoxamine, paroxetine, sertraline, desmethylamitriptyline, clomipramine, doxepin, imipramine, imipramine oxide, trimipramine, adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norclolipramine, noxiptilin, opipramol, perlapine, pizotyline, propizepine, quinupramine, reboxetine, atomoxetine, bupropion, reboxetine, tomoxetine, duloxetine, milnacipran, mirtazapine, nefazodone, venlafaxine, amiflamine, iproniazid, isocarboxazid, M-3-PPC (Draxis), moclobemide, pargyline, phenelzine, tranylcypromine, vanoxerine, bazinaprine, befloxatone, brofaromine, cimoxatone, clorgyline, adinazolam, alaproclate, amitriptyline/chlordiazepoxide combination, atipamezole, azamianserin, bazinaprine, befuraline, bifemelane, binodaline, bipenamol, caroxazone, cericlamine, cianopramine, cimoxatone, clemeprol, clovoxamine, dazepinil, deanol, demexiptiline, dibenzepin, droxidopa, enefexine, estazolam, etoperidone, femoxetine, fengabine, fezolamine, fluotracen, idazoxan, indalpine, indeloxazine, iprindole, levoprotiline, lithium, litoxetine, lofepramine, medifoxamine, metralindole, mianserin, milnacipran, minaprine, mirtazapine, montirelin, nebracetam, nefopam, nialamide, nomifensine, norfluoxetine, orotirelin, oxaflozane, pinazepam, pirlindone, pizotyline, ritanserin, rolipram, sercloremine, setiptiline, sibutramine, sulbutiamine, sulpiride, teniloxazine, thozalinone, thymoliberin, tianeptine, tiflucarbine, trazodone, tofenacin, tofisopam, toloxatone, tomoxetine, veralipride, viloxazine, viqualine, zimelidine, and zometapine.

By “anticonvulsive” is meant any of a diverse group of agents used in prevention of the occurrence of epileptic seizures (i.e., antiepileptic). The goal of an anticonvulsant is to suppress the rapid and excessive firing of neurons that start a seizure. Many anticonvulsants block sodium (Na⁺) channels, calcium (Ca²⁺) channels, AMPA receptors, or NMDA receptors. Some anticonvulsants inhibit the metabolism of GABA or increase its release. Examples of anticonvulsants include, but are not limited to, carbamazepine, flupirtine, gabapentin, lamotrigine, oxcarbazepine, phenyloin, retigabine, topiramate, and valproate.

By “anxiolytic” is meant an agent that is used to reduce the symptoms of anxiety. A class of anxiolytics is the benzodiazepines that include, but are not limited to, lorazepam, clonazepam, alprazolam and diazepam. Antidepressants such as selective serotonin reuptake inhibitors (SSRIs) may also be anxiolytic.

By “cyclooxygenase-2 (COX-2) inhibitor” is meant an agent that inhibits the activity of a COX-2 enzyme. Examples of COX-2 inhibitors include, but are not limited to NSAIDS, paracetamol (acetaminophen), celecoxib, etoricoxib, lumiracoxib, parecoxib, rofecoxib, and valdecoxib.

By “non-steroidal anti-inflammatory drug” (NSAID) is meant an agent that exhibits analgesic, anti-inflammatory, and antipyretic effects on a treated subject. Examples of NSAIDS include, but are not limited to, aspirin, amoxiprin, benorilate, choline magnesium salicylate, faislamine, methyl salicylate, magnesium salicylate, salicyl salicylate (salsalate), aceclofenac, bromfenac, etodolac, sulindac, carprofen, fenbufen, loxoprofen, oxaprozin, azapropazone, sulfinpyrazone, nimesulide, licofelone acemetacin, celecoxib, deracoxib, diclofenac, diflunisal, ethenzamide, etofenamate, etoricoxib, fenoprofen, flufenamic acid, flurbiprofen, lonazolac, lomoxicam, ibuprofen, indomethacin, isoxicam, kebuzone, ketoprofen, ketorolac, naproxen, nabumetone, niflumic acid, sulindac, tolmetin, piroxicam, meclofenamic acid, mefenamic acid, meloxicam, metamizol, mofebutazone, oxyphenbutazone, parecoxib, phenidine, phenylbutazone, piroxicam, propacetamol, propyphenazone, rofecoxib, salicylamide, suprofen, tiaprofenic acid, tenoxicam, valdecoxib, 4-(4-cyclohexyl-2-methyloxazol-5-yl)-2-fluorobenzenesulfonamide, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, and 2-(3,5-difluorophenyl)-3-[4-(methylsulfonyl)phenyl]-2-cyclopenten-1-one).

As used herein, the term “opiate” refers to an agent, natural or synthetic, that exerts an analgesic effect upon binding to an opiate receptor in the central nervous system. Examples of opiates include, but are not limited to, alfentanil, butorphanol, buprenorphine, codeine, dextromoramide, dextropropoxyphene, dezocine, dihydrocodeine, diphenoxylate, etorphine, fentanyl, hydrocodone, hydromorphone, ketobemidone, levorphanol, levomethadone, methadone, meptazinol, morphine, morphine-6-glucuronide, nalbuphine, naloxone, oxycodone, oxymorphone, pentazocine, pethidine, piritramide, remifentanil, sulfentanyl, tilidine, tapentadol, and tramadol.

By “pharmaceutically acceptable carrier” is meant a carrier which is physiologically acceptable to the treated human and retains the therapeutic properties of the compound with which it is administered. One exemplary pharmaceutically acceptable carrier is physiological saline. Other physiologically acceptable carriers and their formulations are known to one skilled in the art and are described, for example, in Remington: The Science and Practice of Pharmacy, (21^{st} ed.) ed. A. R. Gennaro, 2006, Mack Publishing Company, Easton, Pa. and Encyclopedia of Pharmaceutical Technology, (3^{rd} ed.) ed. J. Swarbrick, 2006, Marcel Dekker, New York, which is incorporated herein by reference.

By “stimulus” is meant an agent or action that induces a physiological or psychological activity or response. For example, a chemical stimulus includes one or more chemicals that are capable of affecting an animal. A chemical stimulus can include an inflammatory composition. A mechanical stimulus includes any action involving physical contact with the animal that is capable of affecting the animal, e.g., applying pressure to a part of the animal. A tactile stimulus includes any stimulus that involves the sense of touch of the animal being stimulated, e.g., a mechanical stimulus of the skin. A control stimulus is a stimulus that induces a known response from the animal being stimulated. For example, a control stimulus can be a stimulus that causes a minimal effect and is used as a negative control for purposes of comparison to the effect caused by a test stimulus.

By “triptan” is meant a tryptamine-based drug that binds to serotonin 5-HT_{1B} and 5-HT_{1D} receptors and promotes inhibition of pro-inflammatory neuropeptide release. Triptans are a diverse family of drugs commonly used in the treatment of migraine and headaches. Examples of triptans include, but are not limited to, sumatriptan, rizatriptan, naratriptan, zolmitriptan, eletriptan, almotriptan, and frovatriptan.

By an “effective amount” is meant an amount sufficient to achieve a desirable therapeutic or prophylactic result in a subject.

The term “therapeutic” refers to an agent, dosage, or treatment that is ameliorative or curative in nature; that may diminish the duration, frequency, or severity of any discomfort or pain, or physical limitations associated with recuperation from a disease, disorder, or physical trauma involving visceral pain; or that may be used as an adjuvant to other therapies and treatments for conditions involving visceral pain.

The term “prophylactic” refers to an agent, dosage, or treatment that is preventive or pre-emptive, e.g., treatment following an event expected to result in visceral pain, and encompasses procedures designed to target individuals at risk of suffering from visceral pain.

By “visceral pain” is meant any pain felt by a subject secondary to a disease, disorder, or condition of an internal organ. Conditions that result in visceral pain include, but are not limited to, irritable bowel syndrome, inflammatory bowel syndrome, pancreatitis, diverticulitis, Crohn's disease, peritonitis, pericarditis, hepatitis, appendicitis, colitis, cholecystitis, gastroenteritis, renal pain, interstitial cystitis, ovarian (e.g., cysts), endometriosis, dysmenorrhea, uterine pain, pain resulting from a cancer of a visceral organ, pain from injury, infection of an internal organ, gynecological pain, bladder pain, bowel pain, stomach pain, esophageal pain, referred cardiac pain, upper gastrointestinal dyspepsia, and colic (including renal and biliary colic). Visceral pain can be experienced by any animal with a disease or condition of any internal organ.

Other features and advantages will be apparent from the following description and the claims.

### BRIEF DESCRIPTION OF THE DRAWINGS

FIGS. 1A-1D are graphs that show the effects of systemic sumatriptan in experimental visceral pain in rats. FIG. 1A: Time course of the effects of sumatriptan in rats with pancreatitis (DBTC) or without pancreatitis (vehicle). Sumatriptan attenuated the frequency of withdrawals of DBTC-treated rats within 20 min of administration, with peak effect at 40 min in a dose-dependent manner. FIG. 1B: Dose-response curve of sumatriptan 40 min after intraperitoneal (IP) injection in rats with experimental pancreatitis. Sumatriptan reduced abdominal withdrawals in pancreatic rats in a dose-dependent manner. FIG. 1C: Time course of the effects of sumatriptan in rats with colonic hypersensitivity (butyrate) or controls (saline). Systemic (IP) administration of sumatriptan reversed the reduction of mechanical threshold in butyrate-treated rats within 20 min of administration, with the peak effect at 40 min post-administration. FIG. 1D: Dose-response curve of sumatriptan 40 min after IP injection in rats with experimental colonic hypersensitivity (n=8 per dose). Systemic sumatriptan restored tactile thresholds of butyrate-treated rats in a dose dependent manner.

FIGS. 2A and 2B are graphs showing the effect of systemic serotonin agonists on the effect of systemic sumatriptan. FIG. 2A: In rats with experimental pancreatitis (DBTC-treated), sumatriptan (300 μg/kg; IP) attenuated the frequency of withdrawals compared with rats receiving IP saline (^{#}p<0.05 v. saline group). The 5HT_{1B} antagonist isamoltane (4 mg/kg; IP) reduced the effects of sumatriptan. The 5HT_{1D} antagonist BRL15722 (0.3 μg/kg; IP) also reduced the effects of systemic sumatriptan (*p<0.05 v. sumatriptan group). FIG. 2B: In rats with experimental colonic hypersensitivity (butyrate-treated), sumatriptan (300 μg/kg; IP) increased the mechanical threshold compared with rats receiving saline (#p<0.05 v. saline group). The 5HT_{1B} antagonist isamoltane (4 mg/kg; IP) reduced the effects of sumatriptan. The 5HT_{1D} antagonist BRL1 5722 (300 μg/kg; IP) also reduced the effects of systemic sumatriptan. (*p<0.05 v. control group with no colonic hypersensitivity; n=8 per experimental group).

FIGS. 3A-3D are graphs showing the effect of microinjection of sumatriptan into the RVM in experimental visceral pain in rats. FIG. 3A: Time course of the effects of RVM sumatriptan in rats with pancreatitis (DBTC) or without pancreatitis. Sumatriptan attenuated the frequency of withdrawals of DBTC-treated rats within 20 min of administration, with peak effect at 40 min and diminished effect at the 60 min mark. FIG. 3B: Dose-response curve of sumatriptan 40 min after microinjection in the RVM of rats with experimental pancreatitis. Sumatriptan reduced the number of withdrawals in a dose-dependent manner. FIG. 3C: Time course of the effects of RVM sumatriptan in rats with colonic hypersensitivity (butyrate) or in controls (saline). Sumatriptan reversed the reduction of mechanical threshold in butyrate-treated rats within 20 min of administration, with the peak effect at 40 min post-administration and diminished effect at the 60 min mark. FIG. 3D: Dose-response curve of sumatriptan 40 minutes after microinjection into the RVM of rats with experimental colonic hypersensitivity (n=8 per dose). Systemic sumatriptan restored tactile thresholds of butyrate-treated rats in a dose dependent manner.

FIGS. 4A and 4B are graphs showing the effect of RVM serotonin antagonists on the antinociceptive effects of RVM sumatriptan. FIG. 4A: In rats with experimental parcreatitis (DBTC-injected), sumatriptan (10 μg) microinjected in the RVM attenuated the frequency of withdrawals compared with rats receiving saline in the RVM (#p<0.05 v. saline group). The 5HT_{1B} antagonist isamoltane (3 μg) blocked the effects of sumatriptan (*p<0.05 v. control group with no pancreatitis). The 5HT_{1D} antagonist BRL1 5722 (3 μg) did not have any effect. FIG. 4B: In rats with experimental colonic hypersensitivity (butyrate-treated), sumatriptan (10 μg) microinjected in the RVM increases the mechanical threshold compared with rats receiving saline in the RVM (#p<0.05 v. saline group). The 5HT_{1B} antagonist isamoltane (3 μg) blocked the effects of sumatriptan (*p<0.05 v. control group with no colonic hypersensitivity; n=8 per experimental group). The 5HT_{1D} antagonist BRL1 5722 (3 μg) did not have any effect.

FIGS. 5A and 5B are graphs showing the effect of serotonin antagonists microinjected in the RVM on the effect of systemic sumatriptan. FIG. 5A: In rats with experimental pancreatitis (DBTC-treated), sumatriptan (300 μg/kg; IP) attenuated the frequency of withdrawals compared with rats receiving saline (#p<0.05 v. saline group). The 5HT_{1B} antagonist isamoltane (3 μg) in the RVM failed to antagonize the effects of systemic sumatriptan. The 5HT1_{1D} antagonist BRL1 5722 (3 μg) in the RVM failed to antagonize the effects of systemic sumatriptan. FIG. 5B: In rats with experimental colonic hypersensitivity (butyrate-treated), sumatriptan (300 μg/kg; IF) increased the mechanical threshold compared with rats receiving saline (#p<0.05 v. saline group). The 5HT_{1B} antagonist isamoltane (3 μg) in the RVM failed to antagonize the effects of systemic sumatriptan. The 5HT_{1D} antagonist BRL15722 (3 μg) failed to antagonize the effects of systemic sumatriptan (*p<0.05 v. control group with no colonic hypersensitivity; n=8 per experimental group).

### DETAILED DESCRIPTION OF THE INVENTION

The present invention features methods of treating visceral pain in a human with 5HT_{1B} or 5HT_{1D} receptor agonists, or co-administration of these agents with analgesic, antidepressant, or anticonvulsant drugs. Agonists of 5HT_{1B/1D} receptors that may be useful in the invention include antidepressants, amphetamines, antiemetics, anxiolytics, and triptans (e.g., sumatriptan).

### Visceral Pain

Pain affecting the visceral organs is extremely common and can be severe. Injury and inflammation can be particularly problematic, as organs become highly sensitive to any kind of stimulation, e.g., as in inflammatory bowel disease. Visceral nociceptors respond not only to intense mechanical stimuli (distension and overstretching) but also to irritant chemicals and the products of inflammation. Visceral pain may affect, without limitation, the liver, kidney, ovary, uterus, bladder, bowel, stomach, esophagus, duodenum, intestine, colon, spleen, pancreas, appendix, heart, or peritoneum. Causes of visceral pain include injury, infection, inflammation, chemical irritants, and disease. Conditions commonly associated with visceral pain include irritable bowel syndrome, inflammatory bowel syndrome, pancreatitis, diverticulitis, Crohn's disease, peritonitis, pericarditis, hepatitis, appendicitis, colitis, cholecystitis, gastroenteritis, endometriosis, dysmenorrhea, interstitial cystitis, upper gastrointestinal dyspepsia, renal colic, biliary colic, or infection of a visceral organ.

### 5HT_{1B/1D} Receptors

5HT receptors are present both in the central nervous system and in the periphery where they mediate the effects of endogenous serotonin. For example, peripheral 5HT_{1B} receptors are found in meningeal blood vessels, where sumatriptan is thought to exert its anti-migraine effects (Ahn and Basbaum, Pain 115:1-4 (2005)). Both 5HT_{1B} and 5HT_{1D} receptors have been localized to regions consistent with a role in modulation of visceral pain. First, both 5HT_{1B} and 5HT_{1D} are expressed in the RVM, a region in the brain implicated in modulation of visceral pain (Vera-Portocarrero et al., Gastroenterology 130:2155-2164 (2006)). Moreover, 5HT_{1B} receptors are localized to the gastrointestinal tract and enteric neurons (De Ponti and Tonini, Drugs 61:317-332 (2001)).

As antagonists of 5HT_{1B} or 5HT_{1D} can reverse the ameliorative effects of an agonist of 5HT_{1B} and 5HT_{1D} receptors on visceral pain (see the Examples), agonism of 5HT_{1B/1D} receptors is an operative mechanism for the treatment of visceral pain according to the methods of the invention.

### 5HT_{1B/1D} Receptor Agonists

Agonists of 5HT_{1B/1D} receptors augment activation of the receptors, thereby treating the visceral pain of the human. Accordingly, the methods of the invention feature administration of an effective amount of a 5HT_{1B/1D} receptor agonist. 5HT_{1B/1D} receptor agonists may include antidepressants (e.g., selective serotonin reuptake inhibitors), amphetamines, antiemetics, anxiolytics, anticonvulsants, and triptans. Exemplary 5HT_{1B/1D} receptor agonists are methylphenidate, dolasetron, granisetron, odansetron, tropisetron, palonosetron, lorazepam, clonazepam, alprazolam, diazepam, dolasetron, granisetron, odansetron, tropisetron, palonosetron, gabapentin, vigabatrin, progabide, tiagabine, valproate, carbamazapine, amitriptyline, desipramine, fluoxetine, paroxetine, venlafaxine, sertraline, escitalopram, citalopram, fluvoxamine, milnacipran or duloxetine. Other exemplary 5HT_{1B/1D} receptor agonists include amphetamine, citalopram, dapoxetine, zimelidine, clorazepate, and midazolam. Additional 5HT_{1B/1D} receptor agonists are described herein and known in the art.

### Triptans

Triptans are 5HT_{1B/1D} receptor agonists that may be particularly useful for the treatment of visceral pain. Presently used for abortive treatment of migraine and cluster headaches, triptans are a large family of tryptamine-based drugs that agonize 5HT_{1B} or 5HT_{1D} serotonin receptors. Non-limiting examples of triptans include sumatriptan, rizatriptan, naratriptan, zolmitriptan, eletriptan, almotriptan, and frovatriptan.

Sumatriptan is described in U.S. Pat. No. 4,816,470 and is a widely used triptan for the treatment of migraine. Analogs of sumatriptan may also agonize 5HT_{1B} or 5HT_{1D} receptors and accordingly may be used in certain embodiments of the invention. A large number of sumatriptan analogs have been described in the literature, for example, in U.S. Pat. Nos. 6,255,334, 5,863,935, 5,468,768, 5,466,699, 5,399,574, 5,331,005, 5,270,333, 5,103,020, 5,037,845, 4,994,483, 4,894,387, and 4,816,560. These or other triptans may be useful for the treatment of visceral pain in a human according to methods of the invention.

### Administration and Dosage

In the present invention, pharmaceutical compositions are administered that contain a therapeutically effective amount of a 5HT_{1B} or a 5HT_{1D} agonist. Additional embodiments include one or both agonists with one or more of an analgesic, antidepressant, anxiolytic, antiemetic, amphetamine, or anticonvulsive. The active ingredients thereof may be present in the same pharmaceutical composition (a single dosage form) or in separate pharmaceutical compositions (separate dosage forms) which may be administered concomitantly or at different times. The compositions can be formulated for use in a variety of drug delivery systems. One or more physiologically acceptable excipients or carriers can also be included in the compositions for proper formulation. Suitable formulations for use in the present invention are found, e.g., in Remington: The Science and Practice of Pharmacy, (21^{st} ed.) ed. A. R. Gennaro, 2006, Mack Publishing Company, Easton, Pa. and Encyclopedia of Pharmaceutical Technology, (3^{rd} ed.) ed. J. Swarbrick, 2006, Marcel Dekker, New York. For a brief review of methods for drug delivery, see Langer, Science 249:1527-1533 (1990).

The pharmaceutical compositions are intended for parenteral, intranasal, topical, oral, or local administration, such as by a transdermal means, and for prophylactic and/or therapeutic treatment. Commonly, the pharmaceutical compositions are administered parenterally (e.g., by intravenous, intramuscular, or subcutaneous injection), or by oral ingestion, or by topical application at areas affected or proximal to the site of visceral pain. Intracolonic instillation is another route of administration that may be suitable in certain embodiments of the present invention. Additional routes of administration include intravascular, intra-arterial, intratumoral, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral, oral, and intracolonic administration that comprise the above mentioned agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount of a 5HT_{1B/1D} agonist can be administered for prophylactic and/or therapeutic treatments. In prophylactic applications, compositions are administered to a patient with a clinically determined predisposition or increased susceptibility to visceral pain, or development of a disease that results in visceral pain (e.g., inflammatory bowel disease). Compositions of the invention will be administered to the patient in an amount sufficient to delay, reduce, prevent, or alleviate visceral pain. In therapeutic applications, compositions are administered to a patient already suffering from visceral pain in an amount sufficient to alleviate or at least reduce the pain. An amount adequate to accomplish this purpose is defined as a “therapeutically effective dose.” Amounts effective for this use may depend on the severity of the underlying disease or condition and the weight and general state of the patient, but generally range from about 0.5 mg to about 3000 mg of the agent or agents per dose per patient. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a patient as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood may be employed.

The therapeutically-effective amount of one or more agents present within the compositions of the invention and used in the methods of this invention applied to a human can be determined by the ordinarily-skilled artisan with consideration of individual differences in age, weight, severity of visceral pain, and the condition of the human.

The patient may also receive said agents in the range of about 0.1 to 3,000 mg per dose one or more times per week (e.g., 2, 3, 4, 5, 6, or 7 or more times per week), 0.1 to 2,500 mg per dose per week, 0.1 to 2,000 mg per dose per week, 0.1 to 1,500 mg per dose per week, 0.1 to 1,000 mg per dose per week, 0.1 to 800 mg per dose per week, 0.1 to 600 mg per dose per week, 0.1 to 500 mg per dose per week, 0.1 to 400 mg per dose per week, 0.1 to 300 mg per dose per week, 0.1 to 200 mg per dose per week, 0.1 to 150 mg per dose per week, 0.1 to 100 mg per dose per week, or 0.1 to 50 mg per dose per week. A patient may also receive a 5HT_{1B/1D} agonist of the composition in the range of 0.1 to 3,000 mg per dose once every two or three weeks.

The co-administration of any agents according to the methods of this invention refers to the use of at least two active ingredients in the same general time period or administration of two or more agents using the same general administration method. It is not always necessary, however, to administer both at the same time or in the same way. For instance, if a triptan and an NSAID are administered to a subject suffering from visceral pain in two separate pharmaceutical compositions, the two active agents administered need not be delivered to the patient during the same time period or even during two partially overlapping time periods. In some cases, the administration of the second agent may begin shortly after the completion of the administration period for the first agent or vice versa. Such a time gap between the two administration periods may vary from one day to one week, one month, or longer. In some cases, one therapeutic modality may be administered first with the second in a time period, and subsequently administered without the second in a following period. A typical schedule for this type may require a higher dosage of the first therapeutic modality in the first co-administration period, and a lower dosage in the second period.

Single or multiple administrations of an effective amount of a 5HT_{1B/1D} agonist can be carried out with dose levels and pattern being selected by the treating physician. The dose and administration schedule can be determined and adjusted based on the severity of the visceral pain or underlying condition, which may be monitored throughout the course of treatment according to the methods commonly practiced by clinicians or those described herein.

### EXAMPLES

The following examples are provided for the purpose of illustrating the invention and are not meant to limit the invention in any way.

We examined the effects of sumatriptan in two established rodent models of visceral pain. One model resembles some aspects of pancreatitis by producing inflammation of the pancreas and referred cutaneous hypersensitivity of the abdominal area (Vera-Portocarrero et al., Anesthesiology 98:474-484 (2003)). Pain from pancreatitis can be referred to somatic structures in humans (Buscher et al., Eur J Pain 10:363-370 (2006)) and in animal models (Vera-Portocarrero et al., Anesthesiology 98:474-484 (2003); Winston et al., J Pain 4:329-337 (2003); Wick et al., Am J Physiol Gastrointest Liver Physiol 290:G959-G969 (2006)). Measuring the degree of referred somatic hypersensitivity has become a useful approach to evaluate visceral hypersensitivity and has been applied to study persistent pain associated with inflammation of the pancreas (Dimcev.ki et al., Pancreas 35:22-29 (2007); Dimcev.ki et al., Gastroenterology 132:1546-1556 (2007)). An increase in the area of referred hypersensitivity is also observed in patients with pancreatitis. (Buscher et al., Eur J Pain 10:363-370 (2006)). Recently, a novel model of colonic hypersensitivity has been developed (Bourdu et al., Gastroenterology 128:1996-2008 (2005)) and has been suggested to mimic some aspects of IBS. This model elicits cutaneous hypersensitivity in the lumbar dermatomes of rodents similar to reports of hypersensitivity in patients with IBS (Verne et al., Pain 93:7-14 (2001)). Additionally, this novel model induces hypersensitivity without producing injury or apparent inflammation of the colon, similar to what is seen in patients with IBS (Azpiroz et al., Neurogastroenterol Motil. 19:62-88 (2007)).

In the examples described herein, we explored the actions and mechanisms of triptans in the modulation of visceral pain. For migraine, triptans are thought to act on blood vessels of the meningeal vasculature (Humphrey and Goadsby, Cephalalgia 14:401-410 (1994)) and in the trigeminal ganglion (Ahn and Basbaum, Pain 115:1-4 (2005)). Nonetheless, the receptors upon which sumatriptan exerts its effects are widely expressed in the peripheral nervous systems, suggesting possible activity of the triptans in visceral pain states. In addition to peripheral expression, triptan receptors are found within the central nervous system including areas of pain modulation such as the rostral ventromedial medulla (RVM) (Castro et al., Neuropharmacology 36:535-542 (1997)). Previous studies have implicated the RVM in descending modulation of visceral pain. Electrical stimulation of the RVM produces biphasic modulation of spinal cord responses to acute colorectal distention (Zhuo et al., J Neurophysiol. 87:2225-2236 (2002)) and of colorectal distention-induced nociceptive reflexes (Zhuo et al., Gastroenterology 122:1007-1019 (2002)). Microinjection of lidocaine into the RVM reduced spontaneous activity and responses of spinal neurons to colorectal distention (Zhuo et al., Gastroenterology 122:1007-1019 (2002)). The RVM also has a facilitatory role on persistent visceral pain. Microinjection of lidocaine into the RVM attenuated referred visceral hypersensitivity induced by pancreatic inflammation (Vera-Portocarrero et al., Gastroenterology 130:2155-2164 (2006)).

### Example 1

### Systemic Sumatriptan Reduces Referred Hypersensitivity in Visceral Pain Models

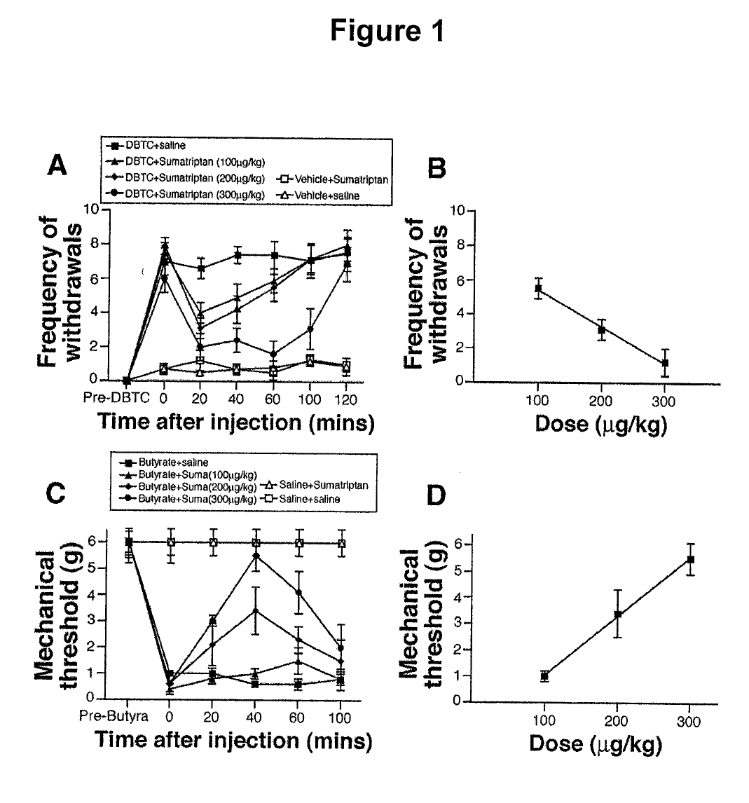

In the experimental pancreatitis model, following IV dibutyltin dichloride (DBTC), rats showed significantly increased withdrawal frequency to mechanical stimulation of the abdomen compared with rats injected with vehicle, indicating development of pancreatitis and associated referred abdominal hypersensitivity as previously described (Vera-Portocarrero et al., Anesthesiology 98:474-484 (2003)) (p<0.05, FIG. 1A, DBTC group treated with saline). On day 6 after IV injection of DBTC, intraperitoneal administration of sumatriptan reduced the frequency of withdrawals in DBTC-injected rats in a time- and dose-dependent manner (FIGS. 1A and 1B). The A50 dose (and 95% confidence interval [CI]) for IP sumatriptan was 172.4 (124.5-386.7) μg/kg. Systemic sumatriptan was active up to 100 minutes postinjection, and the effect dissipated at the 120-minute time point (FIG. 1A). Systemic administration of sumatriptan did not alter responses to abdominal stimulation in vehicle-injected control rats (FIG. 1A). In the colonic hypersensitivity model, rats demonstrated reduced mechanical thresholds when compared with vehicle-treated rats (FIG. 1C, butyrate-saline treated group) indicating the development of referred lumbar hypersensitivity. Systemic sumatriptan increased the mechanical threshold of rats injected with sodium butyrate in a time- and dose-dependent manner (FIGS. 1C and 1D); the A50 (and 95% CI) dose for IP sumatriptan was 232.6 (182.2-322.8) μg/kg. Systemic sumatriptan was active for 60 minutes postinjection, and the effect dissipated by the 100-minute time point (FIG. 1C). Systemic administration of sumatriptan did not modify the behavior of rats previously injected with intracolonic vehicle (FIG. 1C).

### Example 2

### Systemic Actions of Sumatriptan on Visceral Pain Models Are Mediated by Both the 5HT_{1B} and the 5HT_{1D} Receptor

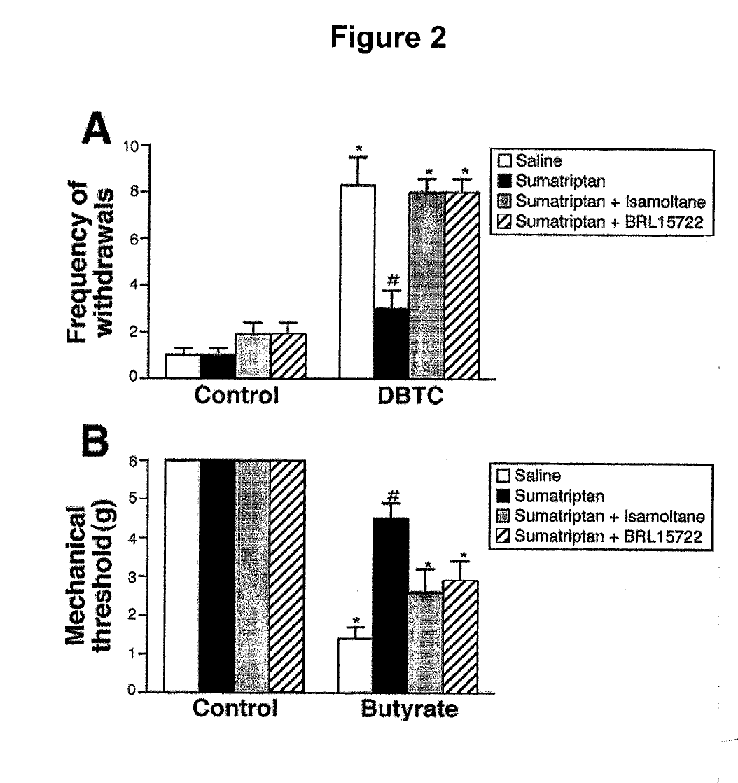

In the experimental pancreatitis model, IV injection of DBTC produced referred abdominal hypersensitivity as indicated by increased frequency of withdrawals (FIG. 2A, DBTC-saline group). As demonstrated above, IP injection of sumatriptan (300 μg/kg) reduced the frequency of withdrawals (FIG. 2A, DBTC-sumatriptan group). Concurrent systemic (IP) injection of the 5HT_{1B} antagonist isamoltane (4 mg/kg) blocked the effects of sumatriptan (p<0.05). Likewise, concurrent IP injection of the 5HT_{1D} antagonist BRL15722 (0.3 mg/kg) with systemic sumatriptan also blocked the effect of sumatriptan (FIG. 2A). The antagonists injected alone did not produce any effects in either vehicle- or DBTC-treated rats (data not shown).

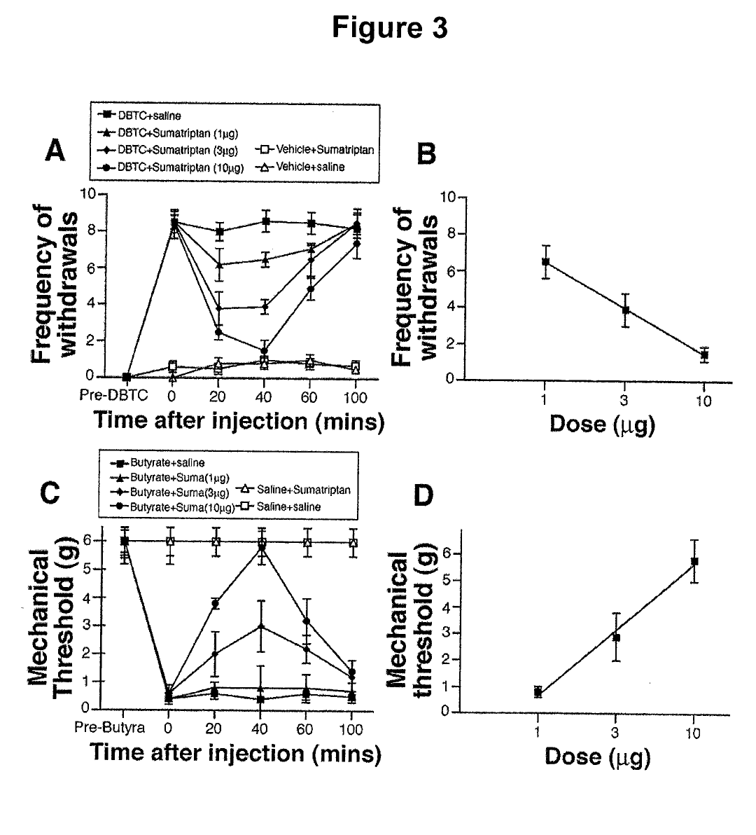

In the colonic hypersensitivity model, colonic injection of sodium butyrate produced referred lumbar hypersensitivity as indicated by a reduction in mechanical threshold to muscle contraction and escape behavior from von Frey stimulation (FIG. 2B, butyrate-saline group). As demonstrated previously, IP injection of sumatriptan (300 μg/kg) increased the mechanical threshold (FIG. 3B, butyrate-sumatriptan group). Concurrent systemic (IP) injection of the 5HT_{1B} antagonist isamoltane (4 mg/kg) blocked the effect of systemic sumatriptan (p<0.05). Likewise, concurrent systemic (IP) injection of the 5HT_{1D} antagonist BRL15722 (0.3 mg/kg) blocked the effect of systemic sumatriptan (FIG. 2B). The antagonists injected alone did not produce any effects in either vehicle- or sodium butyrate-treated rats (data not shown).

### Example 3

### Sumatriptan Acts in the RVM to Reduce Referred Hypersensitivity in Visceral Pain Models

In the experimental pancreatitis model, RVM administration of sumatriptan attenuated the increased frequency of withdrawals associated with referred abdominal hypersensitivity in a time- and dose-dependent manner (FIGS. 3A and 3B). The A50 dose (and 95% CI) for RVM sumatriptan was 4.3 (3.1-16.2) μg. The effects of RVM sumatriptan endured for approximately 60 minutes and dissipated by 100 minutes postinjection (FIG. 3A). Sumatriptan microinjected into the RVM did not alter responses to abdominal stimulation in vehicle-injected rats (FIG. 3A).

In the colonic hypersensitivity model, RVM administration of sumatriptan elicited a time- and dose-dependent attenuation of lumbar hypersensitivity as indicated by an increase in lumbar dermatome mechanical threshold (FIGS. 3C and 3D). The A50 (and 95% CI) dose for RVM sumatriptan was 3.2 (2.0-12.5) μg. The effects of RVM sumatriptan endured for approximately 60 minutes and dissipated by 100 minutes postinjection (FIG. 3C). Microinjection of RVM sumatriptan did not modify the behavior of rats previously injected with intracolonic vehicle (FIG. 3C).

### Example 4

### Sumatriptan Acts through the 5HT_{1B} Receptor in the RVM to Inhibit Referred Hypersensitivity in Visceral Pain Models

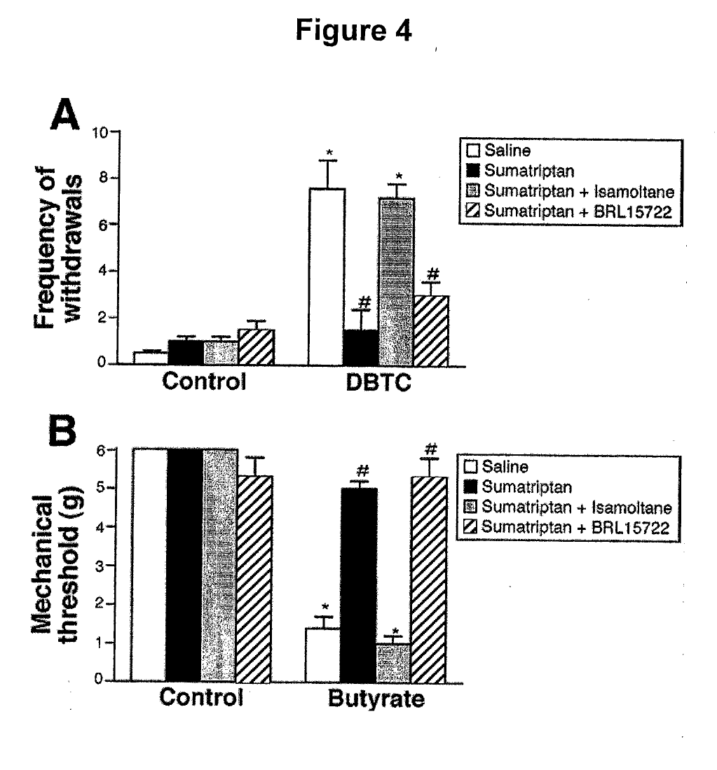

In the experimental pancreatitis model, IV injection of DBTC produced referred abdominal hypersensitivity as indicated by increased frequency of withdrawals (FIG. 4A, DBTC-saline group). RVM microinjection of sumatriptan (10 μg) reduced the frequency of withdrawals in rats with experimental pancreatitis (FIG. 4A, DBTC-sumatriptan group). Concurrent microinjection of the 5HT_{1B} antagonist isamoltane (3 μg) into the RVM blocked the effect of sumatriptan, with the number of withdrawals observed in this group being equivalent to that seen in the saline-treated group (FIG. 4A). In contrast, concurrent microinjection of the 5HT_{1D} antagonist BRL15722 (3 μg) did not block the effect of RVM sumatriptan (FIG. 4A) because the frequency of withdrawals after application of BRL15722 did not differ from the frequency presented by rats receiving RVM sumatriptan alone in rats with pancreatitis. Microinjection of either antagonist alone did not produce any effects in control of pancreatitis rats (data not shown). In the colonic hypersensitivity model, lumbar hypersensitivity as indicated by a reduction in mechanical threshold to muscle contraction and escape behavior from von Frey stimulation was observed (FIG. 4B, butyrate-saline group). Microinjection of sumatriptan (10 μg) into the RVM increased the mechanical threshold (FIG. 4B, butyrate-sumatriptan group).

Concurrent microinjection of the 5HT_{1B} antagonist isamoltane (3 μg) blocked the effect of RVM sumatriptan with observed mechanical thresholds equivalent to the sodium butyrate-treated group (FIG. 4B). In contrast, concurrent microinjection into the RVM of the 5HT_{1D} antagonist BRL15722 (3 μg) did not block the effect of RVM sumatriptan (FIG. 4B). Microinjection of either antagonist alone did not modify the mechanical threshold of rats treated with sodium butyrate (data not shown).

### Example 5

### The RVM is not a Site of Action for Systemically Applied Sumatriptan

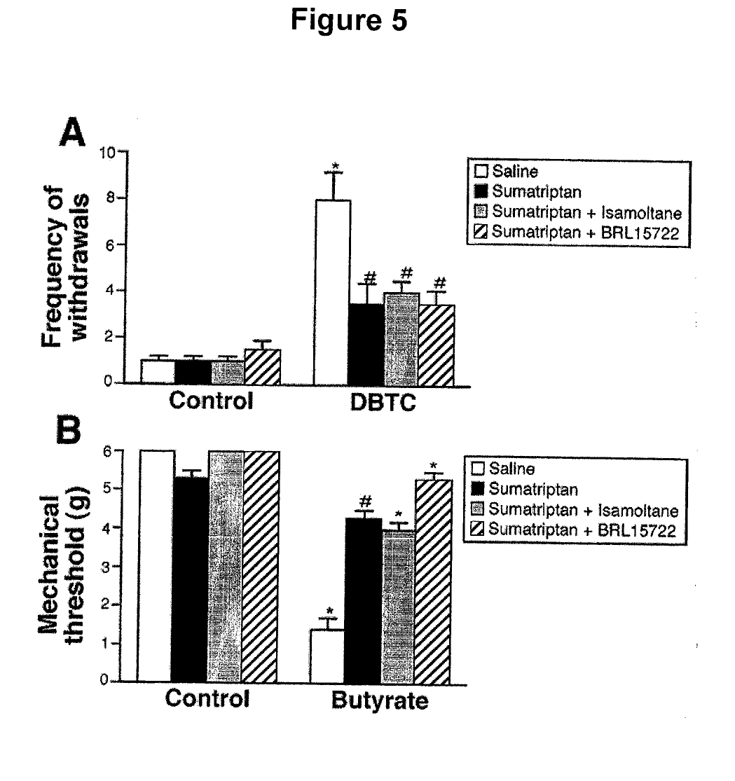

In the experimental pancreatitis model, IV injection of DBTC produced referred abdominal hypersensitivity as indicated by increased frequency of withdrawals (FIG. 5A, DBTC-saline group). As before, IP injection of sumatriptan (300 μg/kg) reduced the frequency of withdrawals (FIG. 5A, DBTC-sumatriptan group). Concurrent microinjection of the 5HT_{1B} antagonist isamoltane (3 μg) into the RVM did not modify the effect of systemic sumatriptan. Likewise, concurrent microinjection of the 5HT_{1D} antagonist BRL15722 (3 μg) with systemic sumatriptan did not block the effect of sumatriptan (FIG. 5A).

In the colonic hypersensitivity model, colonic injection of sodium butyrate produced referred lumbar hypersensitivity as indicated by a reduction in mechanical threshold to muscle contraction and escape behavior from von Frey stimulation (FIG. 5B, butyrate-saline group). As above, IP injection of sumatriptan (300 μg/kg) increased the mechanical threshold (FIG. 5B, butyrate-sumatriptan group). Concurrent microinjection of the 5HT_{1B} antagonist isamoltane (3 μg) into the RVM did not modify the effect of systemic sumatriptan. Likewise, concurrent microinjection into the RVM of the 5HT_{1D} antagonist BRL15722 (3 μg) did not block the effect of systemic sumatriptan (FIG. 5B). As an additional control, rats received IV injection of the highest dose of sumatriptan tested in the RVM (10 μg) and were monitored for signs of referred hypersensitivity (data not shown). Rats with previous injection of DBTC to induce pancreatitis presented increased frequency of abdominal withdrawals, which was not altered by IV injection of 10 μg sumatriptan at any of the time points investigated (up to 120 minutes postinjection). Similarly, rats with previous injection of sodium butyrate presented with reduced threshold to respond to mechanical stimulation of the lumbar dermatomes, and IV sumatriptan (10 μg) did not reverse the reduction in threshold at any of the time points investigated (up to 120 minutes postinjection).

These examples demonstrate that (1) both systemic and RVM administration of sumatriptan significantly inhibit the referred somatic hypersensitivity observed after induction of pancreatitis or colonic hypersensitivity; (2) within the RVM, sumatriptan mediates its antihypersensitivity effects through selective activity of the 5HT_{1B} receptor; and (3) systemic application of sumatriptan blocks referred somatic hypersensitivity from visceral pain through activity at peripheral 5HT_{1B} and 5HT_{1D} receptors. These studies demonstrate that sumatriptan is (1) active in the modulation of inflammatory and noninflammatory visceral pain and (2) can exert antihyperalgesic actions within the RVM in modulation of pain. These findings reveal a novel application and multiple sites and mechanisms of action for triptans in the modulation of visceral pain.

It has been suggested that triptans act exclusively in the trigeminal system to abort migraine pain (Ekbom, Cephalalgia 15 (Suppl 15):33-36 (1995)). However, a few studies suggest that sumatriptan might have activity in other pain states. Thermal hypersensitivity induced by intraplantar injection of carrageenan is attenuated by sumatriptan injected systemically at similar doses used in the present study (Bingham et al., Exp Neurol. 2001 167:65-73 (2001)). Sumatriptan also inhibits capsaicin-induced hyperemia in the sciatic nerve (Zochodne and Ho, Neurology 44:161-163 (1994)), and the evoked release of calcitonin gene-related peptide from the rat isolated spinal cord (Arvieu et al., Neuroreport 7:1973-1976 (1996)). Interestingly, sumatriptan also has antinociceptive efficacy in acetic acid-induced abdominal writhing in mice (Ghelardini et al., Int J Clin Pharmacol Res. 36:1973-1976 (1997); Jain et al., Indian J Exp Biol. 36:973-979 (1998)), which has a visceral pain component. These studies suggest that the triptans might be effective in treatment of a broader spectrum of pain states besides headache pain.

One of the main characteristics of visceral pain is that it is referred to somatic dermatomes receiving innervation from the same areas of the central nervous system that innervate visceral structures (Giamberardino, J Rehabil Med. 85-88 (2003)). Such referred hypersensitivity can be reproduced in animal models of visceral pain (Vera-Portocarrero et al., Anesthesiology 98:474-484 (2003); Winston et al., J Pain 4:329-337 (2003); Wick et al., Am J Physiol Gastrointest Liver Physiol 290:G959-G969 (2006); Al-Chaer et al., Gastroenterology 119:1276-1285 (2000)). In the present examples, we measured referred somatic hypersensitivity in two established models of persistent visceral pain. Experimental pancreatitis is an inflammatory, persistent visceral pain state that is characterized by referred abdominal hypersensitivity that can be measured by frequency of withdrawals to stimulation with von Frey filaments. Enhanced responsiveness in this model is inhibited by opiates (Vera-Portocarrero et al., Anesthesiology 98:474-484 (2003)), NK-1 antagonists (Vera-Portocarrero and Westlund, Pharmacol Biochem Behav. 77:631-640 (2004)), and manipulations that interfere with descending facilitation originating in the RVM (Vera-Portocarrero et al., Gastroenterology 130:2155-2164 (2006)). The second model we used is a recently established model of noninflammatory colonic hypersensitivity, which appears to mimic some aspects of IBS. One of the main characteristics of this model is the development of somatic hypersensitivity in the absence of inflammation of the colon, which is referred to the lumbar dermatomes (Bourdu et al., Gastroenterology 128:1996-2008 (2005)). Somatic hypersensitivity was used as an indication of ongoing persistent visceral pain.

Peripheral 5HT_{1B} receptors have been shown to be present in meningeal blood vessels, where sumatriptan is thought to perform its anti-migraine effects (Ahn and Basbaum, Pain 115:1-4 (2005)). The 5HT_{1D} receptor is usually found in primary afferent terminals of the trigeminal system (Potrebic et al., J Neurosci 23:10988-10997 (2003)) and also in primary afferent terminals in the spinal cord (Ahn and Basbaum, J Neurosci. 26:8332-8338 (2006)). It is thought that sumatriptan acting at these receptors inhibits the release of neurotransmitters (Ahn and Basbaum, Pain 115:1-4 (2005)). The 5HT_{1B} receptor has been localized to the gastrointestinal tract and enteric neurons (De Ponti and Tonini, Drugs 61:317-332 (2001)), but its presence in the pancreas is unknown. One possibility is that this receptor is found in the vasculature in the pancreas, similar to its localization in other organ systems. Regulation of vasculature contractility has a role in the maintenance of pancreatic inflammation and subsequent pain (Bornman et al., World J Surg. 27:1175-1182 (2003)). The 5HT_{1D} receptor is localized in trigeminal afferents where it inhibits release of neurotransmitters (Jennings et al., Pain 111:30-37 (2004); Levy et al., Proc Natl Acad Sci USA 101:4274-4279 (2004)). It is also found in primary afferent terminals in the spinal cord and cell bodies of the dorsal root ganglia (DRG) (Ahn and Basbaum, J Neurosci. 26:8332-8338 (2006)). Sumatriptan may act at this receptor to block the release of neurotransmitter and therefore block the transmission of noxious information (the mechanical stimulation).

Microinjection of sumatriptan into the RVM attenuated referred abdominal hypersensitivity through activity at the 5HT_{1B} receptor, but not the 5HT_{1D} receptor. Both 5HT_{1B} and 5HT_{1D} mRNA have been observed in the RVM (Bruinvels et al., Naunyn Schmiedebergs Arch Pharmacol. 33:367-386 (1993)). Moreover, 5HT_{1B} receptor binding sites have been reported in the RVM (Castro et al., Neuropharmacology 36:535-542 (1997)). The present examples show that the anti-hypersensitivity effects of RVM sumatriptan was blocked by concurrent microinjection of the 5HT_{1B} receptor antagonist isamoltane, but not microinjection the 5HT_{1D} receptor antagonist BRL15722, into the RVM. Thus, it appears that sumatriptan can act centrally at the RVM to attenuate visceral pain through activation of 5HT_{1B} receptor. Further studies are needed to determine localization of 5HT_{1B} and 5HT_{1D} receptors within the RVM, particularly in relation to known descending pain facilitatory and inhibitory cells. Nonetheless, the examples described herein provide more evidence for the concept that sumatriptan has sites of action other than the trigeminal system, as has been demonstrated for the peri-aqueductal gray (PAG) (Bartsch et al., Ann Neurol. 56:371-81 (2004)).

The 5HT_{1D} receptor is localized in trigeminal afferents where its activation results in inhibition of neurotransmitter release (Jennings et al., Pain 111:30-37 (2004), Levy et al., Proc Natl Acad Sci USA 101:4274-4279 (2004)) and is also found in primary afferent terminals in the spinal cord and cell bodies of the dorsal root ganglia (Ahn and Basbaum, J Neurosci. 26:8332-8338 (2006)). Thus, triptans could act at this receptor and block neurotransmitter release and the transmission of evoked noxious stimuli. This concept warrants further study in the context of visceral pain states.

Our previous studies have demonstrated that enhanced descending facilitation arising in the RVM plays an important role in the maintenance of persistent visceral pain (Vera-Portocarrero et al., Gastroenterology 130:2155-2164 (2006)). Like the activity observed following IP sumatriptan, RVM microinjection produced both dose- and time-related antihyperalgesic actions in both models of persistent visceral pain. To confirm that the observed actions were within the RVM and not the result of the RVM injection gaining access to the general circulation, we demonstrated that IV injection of the highest effective dose of RVM sumatriptan failed to attenuate the visceral hypersensitivity in both models (data not shown). These data suggest that the RVM is a potential site of action for sumatriptan in persistent visceral pain. Both 5HT_{1B} and 5HT_{1D} messenger RNA have been observed in the RVM (Bruinvels et al., Neuropharmacology 33:367-386 (1994)), and 5HT_{1B} receptor-binding sites have been reported in the RVM (Castro et al., Neuropharmacology 36:535-542 (1997)). Our data show that the antinociceptive effect of RVM sumatriptan was blocked by concurrent microinjection of the 5HT_{1B} receptor antagonist isamoltane, and not by micro-injection of the 5HT_{1D} receptor antagonist BRL15722. In our study, we used antagonists at doses known to block their respective receptors with high affinity using in vitro assays (Renyi et al., Naunyn Schmiedebergs Arch Pharmacol. 343:1-6 (1991); Price et al., Naunyn Schmiedebergs Arch Pharmacol. 356:312-320 (1997)). Although isamoltane has been reported to have activity at β-2 adrenergic receptors, the possibility that this mechanism may mediate the observed antihyperalgesic actions of sumatriptan appears unlikely because of the reported absence of these receptors within the RVM (Nicholas et al.,” Neuroscience 56:1023-1039 (1993); Li et al., J Neurophysiol. 79:583-594 (1998)). It is also possible that BRL15722 might have antagonized the actions of RVM sumatriptan if the antagonist was given at a higher dose. However, interpretation of this result would be difficult because of the possibility of nonselective actions at other 5HT receptors at higher doses.

### METHODS

### Animals

Adult male Sprague Dawley rats (Harlan, Indianapolis, Ind.), weighing 150-200 g, were maintained in a climate-controlled room with ad lib food and water on a 12 hour/12 hour light/dark cycle (lights on at 07:00 hours). All procedures followed the policies of the International Association for the Study of Pain and the NIH guidelines for the handling and use of laboratory animals. Studies were approved by the University of Arizona IACUC.

### Drugs

Dibutyltin dichloride (DBTC) was obtained from Sigma-Aldrich (Milwaukee, Wis.) and dissolved in 100% ethanol to a concentration of 8 mg/kg (Sparmann et al., Gastroenterology. 112:1664-1672 (1997))). Sumatriptan succinate was obtained from Toronto Research Chemicals (ON, Canada) and dissolved in saline to 1, 3, and 10 μg for RVM microinjections and 100, 200, and 300 μg/kg for systemic injections (Kayser et al., Br J Pharmacol. 137:1287-1297 (2002)). The 5HT_{1B} antagonist isamoltane and the 5HT_{1D} antagonist BRL15722 were obtained from Tocris (Elllisville, Mo.). Isamoltane was dissolved in saline to a concentration of 3 μg for RVM microinjection and to a concentration of 4 mg/kg (Ottani et al., Eur J Pharmacol. 497:181-186 (2004)) for system application. BRL15722 was dissolved in 10% DMSO to a concentration of 3 μg for RVM microinjection and to a concentration of 0.3 mg/kg (Ottani et al., Eur J Pharmacol. 497:181-186 (2004)) for systemic application.

### Experimental Design

For the experiments involving microinjection of drugs into the RVM, rats underwent surgeries to implant RVM cannulae. After five days of recovery, rats received either intravenous injection of DBTC to induce pancreatitis, or intracolonic injection of sodium butyrate to induce colonic hypersensitivity. Animals were monitored for development of visceral hypersensitivity on the subsequent days. On day six after either induction of pancreatitis or colonic hypersensitivity, animals underwent baseline behavioral measurements. Sumatriptan was microinjected into the RVM at different doses (separate groups of rats for each dose) and animals were monitored behaviorally for two h after sumatriptan application in the RVM. For experiments investigating the effects of serotonin antagonists, the drugs were microinjected concurrently with injection of sumatriptan and animals were monitored for the subsequent two h. Separate groups of animals were microinjected with the antagonists alone to control for possible effects of the drugs by themselves.

For the experiments involving systemic drug administration, rats received intravenous injection of DBTC to induce pancreatitis or intracolonic injection of sodium butyrate to induce colonic hypersensitivity. On day six after either induction of pancreatitis of colonic hypersensitivity, animals underwent baseline behavioral measurements. Sumatriptan was injected intraperitoneally at different doses (separate groups of rats for each dose) and the animals were monitored behaviorally every 20 min for two h after injection. For experiments investigating the effects of serotonin antagonists, the drugs were injected intraperitoneally immediately following the injection of sumatriptan (separate groups for each respective antagonist). Separate groups of animals were injected with the antagonists alone. Additionally, separate groups of animals were injected with sumatriptan systemically and microinjected with the antagonists in the RVM to determine if the RVM is a site of action for systemically applied sumatriptan. The microinjection of antagonists into the RVM was done immediately following the systemic injection of sumatriptan. All animals were tested for behavioral signs of hypersensitivity every 20 min for two h after the end of the injections.

### Visceral Pain Models

Pancreatitis was produced by a tail vein injection of dibutyltin dichloride (DBTC, Aldrich, Milwaukee, Wis., 0.25 cc) dissolved in 100% ethanol at a dose of 8 mg/kg under isofluorane anesthesia (2-3 liters/min, 4.0%/vol until anesthetized, then 2.5%/vol throughout the procedure; Vera-Portocarrero et al., Gastroenterology. 130:2155-2164 (2006)). Control animals were injected with the vehicle solution only (100% ethanol, 0.25 mL).

Colonic hypersensitivity was induced by enemas of a sodium butyrate solution (1000 mM) twice daily for 3 days (Bourdu et al., Gastroenterology. 128:1996-2008 (2005)). For each enema, a catheter made of P100 polyethylene tube was placed into the colon at 7 cm from the anal opening, and the animals received 1 mL of sodium butyrate at neutral pH. Care was taken to avoid damage of the colonic wall by insertion of the catheter.

### Behavioral Measures

Referred abdominal hypersensitivity in the pancreatitis model was quantified by measuring the number of withdrawal events evoked by application of a calibrated von Frey filament (determined by either abdominal withdrawal, licking of the abdominal area, or whole body withdrawal). Rats were placed inside Plexiglas boxes on an elevated fine fiberglass screen mesh and acclimated for 30 minutes before testing. A 4 g von Frey filament was applied from underneath through the mesh floor, to the abdominal area at different points on the surface. A single trial consisted of 10 applications of this filament applied once every 10 seconds to allow the animals to cease any response and return to a relatively inactive position. The mean occurrence of withdrawal events in each trial is expressed as the number of responses to 10 applications as previously described (Vera-Portocarrero et al., Anesthesiology. 98:474-484 (2003)).

Referred lumbar hypersensitivity in the colonic hypersensitivity model was quantified by applying von Frey hairs to the lumbar dermatomes of rats (Bourdu et al., Gastroenterology. 128:1996-2008 (2005)). Rats were shaved on the lumbar dermatomes before any manipulation and acclimated inside Plexiglas boxes for 30 minutes on the day of testing. Calibrated von Frey hairs of increasing diameter were applied 5 times for 1 second, ranging from 0.04 to 6 g. The mechanical threshold corresponded to the force in grams of the von Frey hair which induced lumbar skin wrinkling followed or not by escape behavior from the filament.

### Surgeries and Microinjection Procedures

Rats were anesthetized with ketamine/xylazine (100 mg/kg) and placed in a stereotaxic headholder. For the RVM cannula implantation procedure, the skull was exposed and two 26-gauge guide cannula separated by 1.2 mm (Plastics One Inc., Roanoke, Va.), were directed at the lateral portions of the RVM (anteroposterior, −11.0 mm from bregma; lateral, −0.6 mm from midline; dorsoventral, −8.5 mm from the cranium and secured to the skull with dental cement as previously described (Burgess et al., J Neurosci 22:5129-5136 2002)). After recovery (5 days), animals were injected with IV DBTC to induce pancreatitis or given intracolonic injections of sodium butyrate to induce colonic hypersensitivity. On day 6 after DBTC injection or initiation of the SB enemas, animals received microinjection of drugs into the RVM. Drug administration, using a Hamilton syringe, was performed slowly expelling 0.5 μl bilaterally of drug solution through a 33 gauge injection needle inserted through the guide cannula and protruding an additional 1 mm into fresh brain tissue to prevent backflow. Animals were tested for referred hypersensitivity every 20 minutes after injection for a period of two hours. Animals were euthanized at the end of the experiments and brain, blood, pancreas and colon were harvested for confirmation of cannula placement in the brain and inflammatory signs in the pancreas and colon.

### Statistical Procedures

Data were analyzed using a 2-factor analysis of variance (ANOVA) followed by the Fisher least significance difference post hoc test to determine differences between experimental groups for the behavioral test across time. One-factor ANOVA was used to detect significant differences in behavioral outcomes within each experimental group over time. A linear regression analysis was used to detect the dose dependency of the effects of sumatriptan and to determine the A50 (the dose producing a 50% response). Significance was established at the p<0.05 level.

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A method of treating visceral pain in a human comprising administering to said human an effective amount of a 5HT_{1B} or 5HT_{1D} receptor agonist.

2. The method of claim 1, wherein said visceral pain is secondary to irritable bowel syndrome, inflammatory bowel syndrome, pancreatitis, diverticulitis, Crohn's disease, peritonitis, pericarditis, hepatitis, appendicitis, colitis, cholecystitis, gastroenteritis, endometriosis, dysmenorrhea, interstitial cystitis, upper gastrointestinal dyspepsia, renal colic, or biliary colic.

3. The method of claim 2, wherein said visceral pain results from pancreatitis.

4. The method of claim 2, wherein said visceral pain results from irritable bowel syndrome.

5. The method of claim 1, wherein said visceral pain is secondary to a disease of the liver, kidney, ovary, uterus, bladder, bowel, stomach, esophagus, duodenum, intestine, colon, spleen, pancreas, appendix, heart, or peritoneum.

6. The method of claim 1, wherein said visceral pain results from a neoplasm, injury, or infection.

7. The method of claim 1, wherein said visceral pain is secondary to an inflammatory disease.

8. The method of claim 1, wherein said visceral pain is secondary to a non-inflammatory disease.

9. The method of claim 1, wherein a 5HT_{1B} receptor agonist and a 5HT_{1D} receptor agonist are co-administered.

10. The method of claim 1, wherein said agonist is a triptan.

11. The method of claim 10, wherein said triptan is sumatriptan, rizatriptan, naratriptan, zolmitriptan, eletriptan, almotriptan, or frovatriptan.

12. The method of claim 11, wherein said triptan is sumatriptan.

13. The method of claim 1, further comprising administering to said human one or more additional agents selected from the group consisting of analgesics, antidepressants, anxiolytics, antiemetics, amphetamines, NOS inhibitors, and anticonvulsants.

14. The method of claim 13, wherein said analgesic is a neurokinin antagonist, CCK antagonist, opiate, paracetamol, or nonsteroidal anti-inflammatory drug (NSAID).

15. The method of claim 14, wherein said NSAID is aspirin, ibuprofen, naproxen, or a selective cyclooxygenase 2 (COX-2) inhibitor.

16. The method of claim 15, wherein said selective COX-2 inhibitor is celecoxib, etoricoxib, lumiracoxib, parecoxib, rofecoxib, or valdecoxib.

17. The method of claim 13, wherein said antidepressant is amitriptyline, desipramine, fluoxetine, paroxetine, venlafaxine, sertraline, escitalopram, citalopram, fluvoxamine, milnacipran, or duloxetine.

18. The method of claim 13, wherein said anticonvulsant is gabapentin, vigabatrin, progabide, tiagabine, valproate, or carbamazapine.

19. The method of claim 13, wherein said anxiolytic is lorazepam, clonazepam, alprazolam, or diazepam.

20. The method of claim 13, wherein said antiemetic is dolasetron, granisetron, odansetron, tropisetron, or palonosetron.

21. The method of claim 13 wherein said amphetamine is methylphenidate.

22. The method of claim 1, wherein said agonist is formulated with a pharmaceutically acceptable carrier.

23. The method of claim 1, wherein said agonist is administered to said human by intracolonic instillation.

24. The method of claim 1, wherein said human has been diagnosed with visceral pain prior to said administering.

25. The method of claim 1, wherein said human is not suffering from a migraine or cluster headache.
